(19) United States
(12) Patent Application Publication
(10) Pub. No.: US 2003/0092773 A1
(43) Pub. Date: May 15, 2003

(54) Process for the separation of the cis trans diasteroisomers of tramadol

(76) Inventor: Graham Evans, Cambridge (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/258,638

(22) PCT Filed: Mar. 19, 2001
(86) PCT No.: PCT/GB01/01194

(30) Foreign Application Priority Data
Apr. 28, 2000 (GB) 0010437.2

(51) Int. Cl. A61K 31/137; C07C 217/34

(52) U.S. Cl. 514/650; 564/339

(57) ABSTRACT

A process for separating a mixture of trans (RR, SS) and cis (RS, SR) tramadol comprises a classical salt resolution using a chiral resolving agent selected from tartaric acid and derivatives thereof, and mandelic acid, provided that the resolving agent is not substantially single enantiomer di-O,O-toluoyltartaric acid.

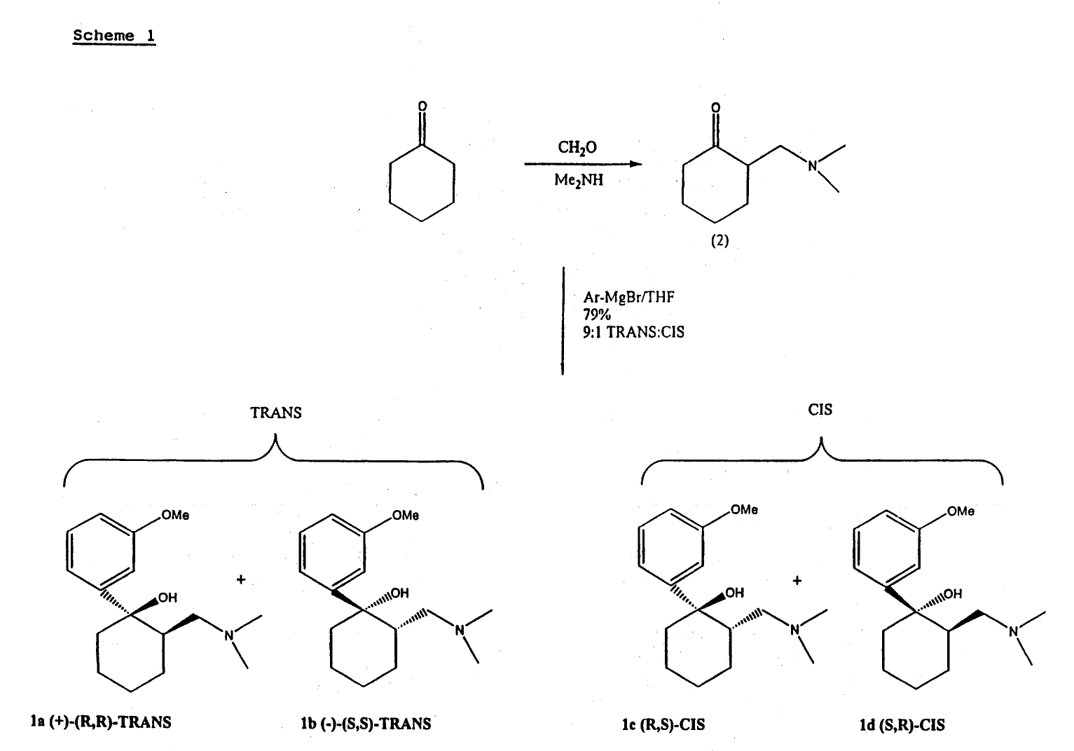

### FIELD OF THE INVENTION

The present invention relates to a process for the separation of the diastereomers of tramadol.

### BACKGROUND TO THE INVENTION

2-[(dimethylamino)methyl]-1-(3-methoxyphenyl)cyclohexanol, is a chiral compound which exists as a mixture of trans (RR,SS) and cis (RS,SR) diastereomers, referred to hereinafter as the trans and cis forms of tramadol, respectively. The trans diastereomer is used as a high-potency analgesic agent.

There is some confusion in the literature as to the nomenclature of the trans and cis forms of tramadol, and further confusion arises as, generally, the name tramadol is used to refer to the substantially pure trans form of the compound. In the context of the present Application, however, the trans and cis forms of tramadol, and the enantiomers thereof, are depicted as 1a to 1d in Scheme 1 below.

Currently, tramadol is typically prepared by the grignard reaction of 3-methoxyphenylmagnesium bromide with 2-[(dimethylamino)methyl]cyclohexanone (2), as shown in Scheme 1 below. This gives rise to a mixture of cis and trans tramadol free base. Separation of this mixture, to obtain the desired trans diastereomer-, can be achieved by a lengthy crystallisation procedure. In the case of U.S. Pat. No. 3,830,934 dioxane is the solvent of choice for this separation. This process is costly due to the long processing time required, and the use of the highly toxic solvent, dioxane (category 1 carcinogen by OSHA), is not ideal.

Other methods of separating the trans diastereomer have been published in the literature. For example, U.S. Pat. No. 5,414,129 crystallises trans tramadol hydrochloride from the grignard mixture in low molecular weight alcohol. However, this process suffers from the need to carry out several crystallisations to remove completely the unwanted cis diastereomer. Likewise U.S. Pat. No. 5,874,620 uses electrophilic reagents, such as acetic anhydride, to react preferentially with the cis diastereomer and crystallise the required trans tramadol from the reaction mixture. Again, further crystallisations are needed to fully remove the cis diastereomer. Another process is disclosed in U.S. Pat. No. 5,877,351, where aqueous hydrogen bromide is used to preferentially crystallise out the trans tramadol hydrobromide. This then requires conversion to the hydrochloride salt.

As reported in our co-pending Application WO-A0032554, it has been found that the substantially single enantiomers of O,O-di-p-toluoyltartaric acid (DTTA) are effective in separating the diastereomers of tramadol, and indeed are capable of separating a single enantiomer of trans tramadol from a mixture of all four possible enantiomers. This provides a more streamlined and economic process than the processes disclosed in the prior art, which does not require initial separation of trans and cis tramadol by a lengthy crystallisation.

### SUMMARY OF THE INVENTION

Surprisingly, it has now been found that O,O-di-ptoluoyltartaric acid is not the only chiral resolving agent which is effective in separating the diastereomers of tramadol. Thus, according to one aspect of the present invention, a process for separating trans (RR,SS) and cis (RS,SR) tramadol comprises a classical salt resolution using a chiral resolving agent selected from tartaric acid and derivatives thereof, and mandelic acid, provided that the resolving agent is not substantially single enantiomer di-O,O-di-p-toluoyltartaric acid.

According to further aspects of the present invention, a process is provided for increasing the diastereomeric excess (DE) of a mixture of trans and cis tramadol, and a process for separating the enantiomers of trans tramadol, as defined in the claims. According to yet another aspect of the present invention, novel diastereomeric salts of tramadol are provided.

### DESCRIPTION OF THE INVENTION

In a process for separating a mixture of trans and cis tramadol, the resolving agent may be used in racemic form or in substantially single enantiomer form.

Tartaric acid and its derivatives have been found to be particularly useful in the present invention. Preferred resolving agents include O,O-dibenzoyltartaric acid (DBTA), tartaric acid (TA) and dibenzoyltartaric acid mono(diethylamide) (DBTAM). Racemic O,O-di-p-toluoyltartaric acid may also be useful in separating the diastereomers of tramadol.

Each of O,O-dibenzoyltartaric acid and tartaric acid has been disclosed in the prior art as effective in separating the enantiomers of trans tramadol. For instance, U.S. Pat. No. 5,723,668 reports the use of L-(+)-tartaric acid to resolve the trans diastereomer. However, as mentioned in WO-A-0032554, in our hands the results reported in U.S. Pat. No. 5,723,668 could not be reproduced. Furthermore, while U.S. Pat. No. 3,830,934 discloses the use of O,O,-dibenzoyl-D-tartaric acid as a resolving agent for trans tramadol, there has been no suggestion that either this resolving agent or tartaric acid would be useful in resolving a mixture of the trans and cis diastereomers.

Another chiral acid that is useful in the present invention is mandelic acid (MAN). Resolution of the enantiomers of trans tramadol using mandelic acid was first disclosed by Elsing et al, Arch. Pharm. (1991) 324,719, without any details of the reaction conditions used. Subsequently, Itov et al, Org. Proc. Res. Dev. (2000)4:291-294, reported that they had failed to repeat the work of Elsing et al. Instead, Itov et al found it necessary to form the mandelate salt, and then crack and reform the salt three times over in order to effect complete separation of the tramadol enantiomers. This is not ideal. Furthermore, there has been no suggestion that mandelic acid may be useful in separating the trans and cis diastereomers of tramadol.

Different results are achieved according to the resolving agent that is used. Where the resolving agent is tartaric acid or O,O-dibenzoyltartaric acid mono(diethylamide), (+/−)-trans tramadol is separated from racemic (+/−)-cis tramadol by crystallisation in the form of a diastereomeric salt, irrespective of whether the resolving agent is used in racemic or enantiomeric form. In the case of tartaric acid, it may be preferred to use L-tartaric acid as the resolving agent, for economic reasons.

The diastereomeric salt obtained, or the racemic trans tramadol free base obtained by cracking that salt, can then be separated into enantiomeric trans tramadol by any of the conventional means, or through the use of one of the other resolving agents described hereinafter.

Each of these resolving agents (i.e. TA and DBTAM) is also useful for increasing the diastereomeric excess of a mixture of trans and cis tramadol.

Like O,O-di-p-toluoyltartaric acid, substantially single enantiomer O,O-dibenzoyltartaric acid is effective in separating a single enantiomer of trans tramadol from a mixture of all four possible enantiomers. When substantially single enantiomer O,O-dibenzoyl-D-tartaric acid is used as the resolving agent (+)-trans tramadol (1a in Scheme 1) is separated from the other three enantiomers. When substantially single enantiomer O,O-dibenzoyl-Ltartaric acid is used as the resolving agent (−)-trans tramadol (1b in Scheme 1) is separated out. When racemic O,O-dibenzoyltartaric acid is used (+/−)-trans tramadol is separated out.

Mandelic acid behaves in a similar fashion to O,O-dibenzoyltartaric acid. If racemic mandelic acid is used, the result is separation of the trans tramadol diastereomers from the cis tramadol diastereomers. If, however, either of the substantially single enantiomers of mandelic acid is used, a single trans tramadol enantiomer may be separated from the total of four possible enantiomers. For instance, use of substantially single enantiomer(−)-mandelic acid as the resolving agent results in the precipitation of the (−)-trans tramadol. (−)-mandelic acid salt, leaving the opposite trans enantiomer in the mother liquors. After two crystallisations, this resolution is capable of obtaining tramadol with an enantiomeric excess of greater than 99%, as compared to the resolution reported by Itov et al, which required more than four crystallisations to achieve this level of optical purity.

As the substantially single enantiomers of each of O,O-dibenzoyltartaric acid and mandelic acid are effective in separating a single enantiomer of trans tramadol from a mixture of all four possible enantiomers, these resolving agents are also useful in processes in which the enantiomers of trans tramadol are to be separated, for instance where the trans and cis tramadol diastereomers have been separated by other means.

According to the present invention it is also possible to use a combination of resolving agents, for instance where this may give rise to an advantage on economic grounds. In particular, mandelic acid is a relatively cheap material as compared to the other resolving agents that we have found to be useful in separating the tramadol diastereomers and/or enantiomers. One possible combination of resolving agents which may be particularly beneficial is that of O,O-di-p-toluoyltartaric acid followed by mandelic acid. For instance, (+)-O,O-di-p-toluoyltartaric acid may be used to resolve diastereomeric or enantiomeric trans tramadol to give a precipitate of the (+)-trans tramadol.(+)-ditoluoyltartaric acid salt. The mother liquors enriched in the (−)-trans tramadol enantiomer may then be cracked and treated with (−)-mandelic acid to give, with seeding, the (−)-trans tramadol.(−)-mandelic acid salt. Other combinations of resolving agents may also be envisaged.

The separation process is extremely simple. In a typical example, crude racemic trans/cis tramadol, i.e. contaminated with the other diastereomer, is mixed with the resolving agent of choice in a suitable organic solvent. If required, the liquid mixture may then be seeded with a diastereomeric salt of tramadol having a counterion derived from the resolving agent of choice. Otherwise crystallisation will proceed spontaneously. When mandelic acid is used as the resolving agent, optimum results are achieved by seeding, and thus seeding is preferred.

Typically, the temperature at which the process is carried out is dependent upon the boiling point of the solvent used. However, usually the process is carried out at a temperature in the range 50 to 100° C., preferably 50 to 80° C., with subsequent cooling to, for instance, around 25° C. or below, for isolation of the resulting precipitate. When the reaction mixture is to be seeded, typically this takes place immediately after mixing tramadol with the resolving agent, so that crystal growth is effected at elevated temperature.

The diastereomeric excess of the precipitated salt can be further enhanced by reslurrying and subsequent reprecipitation. Furthermore, if desired, the other diastereomer, together with the other trans tramadol enantiomer when either substantially single enantiomer O,O-dibenzoyltartaric acid or mandelic acid is used as the resolving agent., may be crystallised out on evaporation of the mother liquors.

After the separation, the diastereomeric salt isolated may be converted to the free base, the hydrochloride salt, or any other pharmaceutically-acceptable salt, as desired, by any of the conventional means.

Essentially, the same procedure is used irrespective of the mixture to be separated or enriched.

In the context of the present Application, by substantially single enantiomer typically we mean that one enantiomer of a chiral compound is in an excess of at least 70% by weight as compared to the other enantiomer, preferably in an excess of at least 90 weight %, and more preferably in an excess of at least 95 weight %.

The findings reported in this Application, coupled with those reported in our co-pending Application WO-A0032554, provide a spectrum of separation technologies allowing one to obtain a single diastereomer or a single enantiomer of tramadol, depending on what is desired for a particular application.

The invention is now further illustrated by the following examples.

### EXAMPLES

### Example

### Resolution of (+/−)-trans/cis tramadol with O,O-dibenzoyl-L-tartaric Acid in iso-propanol

5.0 g of racemic tramadol free base (0.0190 mol), obtained according to the procedure described in U.S. Pat. No. 3,830,934 and shown to consist of the trans and cis tramadol diastereomers in the ratio 82.7:17.3, was taken up in 10 ml of iso-propanol. This yellow coloured solution was added to O,O-dibenzoyl-L-tartaric acid ((L)-(−)-DBTA) 7.15 g (0.0190 mol) in 40 ml of isopropanol at 70° C. A seed sample of

(−)-trans tramadol.(L)-(−)-DBTA was added which effected crystallisation on cooling to 50° C. The resolution was gradually cooled to 25° C. and left to age over a period of approximately fifteen hours.

The copious white precipitate that formed was collected by filtration, washing with 20 ml of isopropanol. This gave after drying 2.50 g (20.6%) of (−)-trans tramadol.di-benzoyl-L-tartaric acid salt with a DE of 90.0% (chiral HPLC) with a trans/cis ratio of 98.2:1.8. This salt was reslurried in 12.5 ml of isopropanol to give 2.11 g of the (−)-trans tramadol containing salt, with a DE of 98.6% in 84.4% yield. MP=146.4-148.1° C. (DSC).

Evaporation of the mother liquors from the resolution gave a slightly coloured oil 9.65 g (79.4%), consisting of a mixture of enriched (+)-trans tramadol.di-benzoyl-LWO tartaric acid salt (DE=44.6%, chiral HPLC) and (+/−)-cis tramadol. di-benzoyl-L-tartaric acid salt, with a trans:cis ratio of 79.4:20.4.

### Example 2

### Resolution of (+/−)-trans/cis Tramadol with (D)-(−)-tartaric Acid in Ethanol/Ethyl Acetate

3.0 g of racemic tramadol free base (0.0114 mol), as described in Example 1, was taken up in 10 ml of absolute ethanol. This yellow coloured solution was added to (D)(−)-tartaric acid 1.71 g (0.0114 mol) in 25 ml of ethylacetate at 75° C. A seed sample of (+/−)-trans tramadol.(D)-(−)-tartaric acid was added, which effected crystallisation on cooling to 40° C. The resolution mixture was gradually cooled to 25° C. and left to age over a period of approximately one and a half hours.

The copious white precipitate that formed was collected by filtration, washing with 10 ml of ethylacetate. This gave after drying 1.58 g (33.5%) of (+/−)-trans tramadol. (D)-(−)-tartaric acid salt with a DE of <5.0% (chiral HPLC), with a trans:cis ratio of 91.7:8.3. This salt was reslurried in 20 ml of ethylacetate to give 1.24 g (78.5% yield) of the (+/−)-trans tramadol containing salt, with a DE of <5.0% and a trans:cis ratio of 94.1:5.9, MP=117.1-129.2° C. (DSC). Evaporation of the mother liquors from the resolution above gave a slightly coloured oil 3.13 g (66.5%).

### Example 4

### Resolution of (+/−)-trans/cis Tramadol with O,O-dibenzoyl-L-tartaric Acid mono(diethylamide) in Ethyl Acetate

5.0 g of racemic tramadol free base (0.0190 mol), obtained according to the procedure described in U.S. Pat. No. 3,830,934 and shown to consist of trans and cis in the ratio 82.7:17.3, was taken up in 45 ml of ethyl acetate. This yellow coloured solution is added to the di-benzoyl-Ltartaric acid mono (diethylamide) ((L)-(−)-DBTAM) 7.32 g (0.0190 mol) in 45 ml of ethyl acetate at 70° C. A seed sample of (−)-tramadol.(L)-(−)-DBTAM was added which effects crystallisation on cooling to 40° C. The resolution was gradually cooled to 25° C., and left to age over a period of approximately two hours.

The copious white precipitate that formed was collected by filtration, washing with 20 ml of ethyl acetate. This gave after drying 2.50 g (20.6%) of (−)tramadol. di-benzoyl-L-tartaric acid mono(diethylamide) with a DE of <5.0% (chiral HLPC), with a ratio of trans:cis of 97.7:2.3. Evaporation of the mother liquors from the resolution above gave a slightly coloured oil 9.65 g (79.4%), of (+)-tramadol. di-benzoyl-L-tartaric acid mono (diethylamide) DE=60.8% (chiral HPLC) and with a trans:cis ratio of 31.8:68.2.

### Example 5

### Resolution of (+/−)-trans/cis Tramadol with (D)-(−)-mandelic Acid in iso-propyl Acetate

6.64 g of racemic tramadol free base (0.0190 mol), obtained according to the procedure described in U.S. Pat. No. 3,830,934 and shown to consist of trans and cis tramadol in the ratio 82.3:17.7, was taken up in 30 ml of iso-propyl acetate. This yellow coloured solution was added to (D))-mandelic acid ((D)-(−)-MAN) 7.32 g (0.0190 mol) in 30 ml of iso-propyl acetate at 70° C. A seed sample of (−)tramadol.(D)-(−)-MAN was added, which effected crystallisation on cooling to 35° C. The resolution was gradually cooled to 15° C., and left to age over a period of approximately 16 hours.

The copious white precipitate that formed was collected by filtration, washing with 10 ml of iso-propyl acetate. This gave, after drying, 3.61 g (35.3%) of (−)tramadol. (D)-(−)-mandelic acid salt with a DE of 93.6% (chiral HPLC), with a ratio of trans:cis of 97.8:2.2.

Evaporation of the mother liquors from the resolution gave a slightly coloured oil 6.61 g (64.7%), of (+)tramadol. (D)-(−)-mandelic acid salt, DE=33.4% (chiral HPLC) and with a trans:cis ratio of 72.6:27.4. This salt was cracked to the free base, of which 4.18 g of a yellow coloured oil was obtained. This was taken up in 40 ml of iso-propyl acetate and to this solution was added 2.42 g of (L)-(+)-mandelic acid at 50° C. Upon cooling to 20° C. and seeding with a sample of (+)-tramadol. (L)-(+)-mandelic acid salt crystallisation was effected. The resolution was cooled to 15° C. and stirred for 16 hours. The precipitate that formed was collected by filtration, washing with 10 ml of iso-propyl acetate. This gave 3.32 g (50.3%) of (+)tramadol.(L)-(+)-mandelic acid salt, DE=90.6% (chiral HPLC) and with a trans:cis ratio of 95.7:4.3.

In the Examples, the trans:cis ratio of tramadol was measured using HPLC on a Phenomenex Luna2 C18′ (100×4.6 mm) 5 μm column. A gradient system was used with detection at 210 nm. The samples were prepared by dissolving −20 mg of the diastereomers in 10 ml of dichloromethane and partitioning between 2N NaOH. The organics were removed and washed with water, then dried over MgSO₄. The dichloromethane was concentrated to dryness, and the resulting samples are made up in 20:80 acetonitrile:water.

DE was measured by chiral HPLC on the cracked salt using a Chiralpak AD (250×4.6 mm) 5 μm column, mobile phase 95:5 iso-propanol:0.1% diethylamide in heptane with a flow rate of 1 ml per minute and detection at 273 nm.

1. A process for separating a mixture of trans (RR,SS) and cis (RS,SR) tramadol comprising a classical salt resolution using a chiral resolving agent selected from tartaric acid and derivatives thereof, and mandelic acid, provided that the resolving agent is not substantially single enantiomer O,O-di-p-toluoyltartaric acid.

2. A process according to claim 1, which utilises a substantially single enantiomer of the resolving agent.

3. A process according to claim 1 or claim 2, wherein the resolving agent is tartaric acid.

4. A process according to claim 1 or claim 2, wherein the resolving agent is O,O-dibenzoyltartaric acid.

5. A process according to claim 4, which utilises O,O-dibenzoyl-L-tartaric acid as the resolving agent.

6. A process according to claim 4, which utilises O,O-dibenzoyl-D-tartaric acid as the resolving agent.

7. A process according to claim 1 or claim 2, which utilises di-benzoyltartaric acid mono(diethylamide) as the resolving agent.

8. A process according to claim 1 or claim 2, which utilises mandelic acid as the resolving agent.

9. A process according to claim 8, which utilises substantially single enantiomer (−)-mandelic acid as the resolving agent.

10. A process according to claim 8, which utilises substantially single enantiomer (+)-mandelic acid as the resolving agent.

11. A process for increasing the diastereomeric excess of a mixture of trans (RR,SS) and cis (RS,SR) tramadol comprising a classical salt resolution using a resolving agent as defined in claim 3 or claim 7.

12. A process for preparing substantially single enantiomer of trans (RR,SS) tramadol comprising carrying out a process as defined in claim 3 or claim 7 to separate racemic trans and racemic cis tramadol, and resolving racemic trans tramadol into its separate enantiomers.

13. A process for separating the enantiomers of trans (RR,SS) tramadol comprising a classical salt resolution using a resolving agent as defined in any of claims 4 to 6 and 8.

14. A process for separating the enantiomers of trans (RR,SS) tramadol comprising a classical salt resolution using mandelic acid as the resolving agent, and which comprises seeding a mixture of trans or cis tramadol and mandelic acid with a tramadol.mandelic acid diastereomeric salt.

15. A process for separating a mixture of trans (RR,SS) and cis (RS,SR) tramadol comprising
i) a first classical salt resolution using as the resolving agent O,O-di-p-toluoyltartaric acid, and separating the precipitate that is formed from the mother liquors;
ii) cracking the mother liquors to form tramadol free base; and
iii) a second classical salt resolution, of the cracked mother liquors using as the resolving agent mandelic acid, and recovering the precipitate that is formed.

16. A process according to claim 15, wherein the second classical salt resolution of step (iii) comprises mixing the mother liquors with mandelic acid and seeding the resultant mixture with a tramadol.mandelic acid diastereomeric salt.

17. A process according to claim 15 or claim 16, which utilises substantially single enantiomer O,O-di-ptoluoyl tartaric acid and substantially single enantiomer mandelic acid in steps (i) and (iii) respectively.

18. A process according to any of claims 15 to 17, wherein in the classical salt resolution of step (i) (+)-O,O-di-p-toluoyltartaric acid is used as the resolving agent to give as the precipitate the (+)-tramadol. (+)-O,O-di-p-toluoyltartaric acid salt, and in the classical salt resolution of step (iii) (−)-mandelic acid is used as the resolving agent to give as the precipitate the (−)-tramadol.(−)-mandelic acid salt.

19. A diastereomeric salt of tramadol having a counterion provided by a resolving agent as defined in claim 1.
